(19) United States
(12) Patent Application Publication
(10) Pub. No.: US 2010/0305412 A1
(43) Pub. Date: Dec. 2, 2010

(54) Device and system for wireless monitoring of the vital signs of patients

(76) Inventors: Mark I. Darrah, Cumming, IA (US); Sean Mahoney, Urbandale, IA (US); Cesar Gradilla, West Des Moines, IA (US); John Elson, San Antonio, TX (US)

Correspondence Address: M. Reza Savari, Esq., 7668 Frederiksen Lane, Dublin, CA, 94568, US

(21) Appl. No.: 12/661,757

(22) Filed: Mar. 22, 2010

Related U.S. Application Data
(60) Provisional application No. 61/210,892, filed on Mar. 23, 2009.

(51) Int. Cl. A61B 5/00

(52) U.S. Cl. 600/301

(57) ABSTRACT

The present invention relates generally to the field of vital signs monitors, and particularly to a device and system for wireless monitoring of the vital signs of a plurality of patients simultaneously yet independently. The present invention is particularly adaptable for use by medical emergency personnel or medics in any setting, such as road accident, disaster sites, combat zones, or hospitals. The systems and methods of the present invention allows the monitoring of a plurality of patients by a single health care professional providing more effective monitoring and care for a large number of patients at any one time, and retaining all the information for more effective later triage and decision making by health care providers.

### RELATED APPLICATIONS

The present application claims benefit of priority to U.S. provisional patent application Ser. No. 61/210,892, filed Mar. 23, 2009, which is incorporated by reference herein in its entirety.

### FIELD OF INVENTION

The present invention relates generally to the field of vital signs monitors, and particularly to a device and system for wireless monitoring of the vital signs of a plurality of patients automatically and simultaneously, e.g., far forward to the point of wounding and/or injury.

### BACKGROUND OF THE INVENTION

Monitoring of vital signs of a patient is routinely performed by health care professionals, especially emergency health care professionals for detecting and anticipating medical problems. Vital signs can be measured and monitored in a wide variety of medical settings including at the site of a medical emergency, disaster site, in a combat zone, at the point of injury, in route to further treatment, during treatment at a care facility, or during perioperative or rehabilitation of patients. Vital signs are measurements of the body's most basic physiological and performance functions. Ongoing changes in those vital signs is considered by health care professionals to be as, or more, important than the actual value at any moment. The main vital signs routinely monitored by medical professionals and healthcare providers include body temperature, pulse rate, blood oxygen level, respiration rate, and blood pressure. Depending of the circumstance and status of a patient, other vital signs may be measured or monitored by health care professionals such as the electrical activity of the heart by an electrocardiogram, pulse integrity, skin humidity, the Glascoe Coma Score (GCS), and level of carbon dioxide in expired gases at the end of a respiration cycle (end tidal CO2). All of these vital signs can be observed, measured, and monitored. Still further, more advanced vital signs would include deriving combined measures such as shock index, pulse pressure, pulse integrity, cardiac cycle complexity or heart rate variability, and a host of evolving indices of multi-parameter statistical representations of the composite set of data obtained. These data will enable the assessment of the level at which an individual is currently functioning. Normal ranges of measurements of vital signs change with age and medical condition. To exacerbate the issue, injured patients characteristically change their vital signs as they either improve or degrade so the change and the rate of change in those parameters over time are sometimes more indicative of that patients overall condition than the parameters measured alone. Often the combination of the current characteristics are combined with the characteristic's historical trendlines to obtain a full understanding of the patients state.

The purpose of recording vital signs historically is to establish a baseline on admission to a hospital, clinic, professional office, or other encounter with a health care provider. Vital signs may be recorded by a nurse, physician, physician's assistant, or other health care professional. A significant amount of data now suggests that the earlier these vital signs are taken, out to as close as possible to the time and place of wounding or injury, and the more information that is recorded about what treatments have already been provided, the better that attending medical personnel will be able to deduce and conclude the proper response or treatment for the injured person. The health care professional has the responsibility of interpreting data and identifying any abnormalities from a person's normal state, and of establishing if current treatment or medications are having the desired effect. Being able to understand the changes in the parameters therefore, and the trending at any moment is significantly better than only a single “upon admission” measure.

Vital signs are usually recorded from once hourly to four times hourly after admission, as required by a person's condition. Or, they are taken by attending EMS or medics at the site of injury and relayed back manually or verbally further along in the treatment chain. Vital signs taken manually by similarly trained professionals and with nearly equivalent experience have been shown to vary significantly on a single patient. Similarly these individuals will likely triage the same patient differently based on manual assessment. The vital signs are recorded and compared with normal ranges for a person's age and medical condition if available. Based on these results, a decision is made regarding further actions to be taken. The gap between the records can be excessive and errors in timing and trends may be overlooked.

Typically, continual vital signs monitoring requires either hands-on human attention or bulky, heavy, complicated, and expensive equipment which typically is impossible to have on hand when and where it is needed. Furthermore, in most hospitals, medical emergency situations, disaster sites, and combat zones, care for numerous patients can be difficult for a finite number of clinicians or medically trained care providers to monitor multiple patients on a continuous basis. Commercially available vital signs monitoring equipment may be helpful in these settings, however, the availability and use of such equipment in emergency situations, or in those instances where a plurality of subjects must be monitored, and decisions based on numerous casualties are made, is rarely feasible or possible with today's medical monitors. While there has been a trend to miniaturize vital signs monitoring equipment that are more effective and helpful to medical emergency personnel on site, further improvements in the effectiveness, portability, and ease of use of vital signs monitoring equipment, as well as the simplicity of wireless connectivity of the equipment is desirable, and the devices and systems for wireless monitoring of patients' vital signs of the present invention addresses the existing problems and provides related solutions and benefits.

### SUMMARY OF THE INVENTION

The present invention relates generally to the field of vital signs monitors, and particularly to a highly mobile device and system for wireless monitoring of the vital signs of a plurality of patients simultaneously. The present invention is particularly adaptable for use by medical emergency personnel in any setting, such as road or industrial accidents, disaster sites, combat zones, battlefield aid stations, or hospitals. The systems and methods of the present invention allows the monitoring of a plurality of patients by a single health care professional providing more effective monitoring, better care, wireless tracking by mobile platforms not on the patient, and care for a large number of patients at any one time from anywhere at any time.

The present invention recognizes that patient care and continuous vital signs monitoring of patients, in particular in disaster areas and combat zones where a large number of injured people with widely variable and dynamic injuries have to be cared for simultaneously, can be made more effective and feasible in a more timely manner by providing patient mounted vital signs monitors to the patients that wirelessly communicate and send data to displays mounted on or carried by emergency medical personnel, sometimes referred to as “user(s)”, such that a plurality of patient mounted monitors can wirelessly communicate with one or more user mounted vital signs systems with or without displays. With such a system, users can monitor the vital signs of a plurality of patients by way of the patient mounted vital signs monitor or by way of the user mounted display allowing a fewer number of emergency medical personnel to care for and continuously monitor a large number of patients. This allows the feasibility of non-medically trained personnel, and those with minimum experience to assess patient's vitals automatically until further medical providers arrive on scene. With such a system the care providers, via wireless connectivity, need not even be within range of the location of the patients yet interact with the care of and disposition of the plurality of patients in near real time from a remote location. This allows for a remote expert to consult in patient disposition across the full list of casualties in near real time without the need to be at or near the site.

One aspect of the present invention includes an apparatus for wireless monitoring of the vital signs of a patient, including a plurality of patient mounted vital signs monitors, each having a plurality of sensors for detecting and measuring the vital signs of a patient; a first display for displaying the patient's vital signs, the display being operatively connected to the plurality of sensors, wherein the display can receive and display the patient vital signs; a first transceiver operatively connected to the patient mounted vital signs monitor, and operable to transmit the patient's vital signs data to a remote transceiver; and a first processing means for processing the detected or measured vital signs and for controlling the operation of at least the first display and the first transceiver; a user mounted processing system and monitor including a second transceiver for wireless connection to the first transceivers of the plurality of the patient mounted vital signs monitors; a second display configured to separately display each of the patients' vital signs data received from the plurality of the patient mounted vital signs monitors; and a second processing means for processing the received vital signs data from the plurality of the patient mounted vital signs monitors and for controlling the operation of at least the second display and the second transceiver; whereby, users can monitor the vital signs of a plurality of patients by way of the patient mounted vital signs monitor or by way of the user mounted display.

### BRIEF DESCRIPTION OF THE DRAWINGS

FIG. 1 shows a schematic diagram outlining the main system schematic and connections of an exemplary embodiment of the wireless vital signs monitor.

FIG. 2 shows a schematic diagram of an exemplary embodiment of a non-invasive blood pressure module connectivity.

FIG. 3 shows a schematic diagram of an exemplary embodiment of a wireless vital signs monitor digital signal processing connections and routing for the unit.

FIG. 4a shows a schematic diagram of an exemplary embodiment of an analog electrocardiographic circuit integrated into the device.

FIG. 4b shows a schematic diagram of an exemplary embodiment of an electrocardiographic isolated power and analog to digital conversion integrated into the device.

FIG. 5 shows a schematic diagram of an exemplary embodiment of an internal device memory design of the wireless vital signs monitor.

FIG. 6 shows a schematic diagram of an exemplary embodiment of a membrane switch layout of the wireless vital signs monitor.

FIG. 7 shows a schematic diagram of an exemplary embodiment of a blood oxygen pulse oximetry design of the wireless vital signs monitor.

FIG. 8 shows a schematic diagram of an exemplary embodiment of an organic light emitting diode (OLED) design integrated into the wireless vital signs monitor.

FIG. 9 shows a schematic diagram of an exemplary embodiment of the main power interface for the wireless vital signs monitor.

FIG. 10 shows a schematic diagram of an exemplary embodiment of a PIC24 interface for the wireless vital signs monitor.

FIG. 11 shows a schematic diagram of an exemplary embodiment of a WiFi and buzzer interface for the wireless vital signs monitor.

FIG. 12 shows a schematic diagram of an exemplary embodiment of a main power board battery charger for the wireless vital signs monitor.

FIG. 13a shows a schematic diagram of an exemplary embodiment of a programming, AUX, and DATA ports schematics for the wireless vital signs monitor.

FIG. 13b shows a schematic diagram of an exemplary embodiment of a battery fuel gage, AUX and DATA ports, and low voltage power schematics for the wireless vital signs monitor.

FIG. 14 shows a schematic diagram of an exemplary embodiment of a graphical user interface (GUI) for remote transceiver units showing how multiple patients can be linked to a single screen for all patients. GUI may include color coding based on user selection of alarm limits.

FIG. 15a shows a schematic diagram of an exemplary embodiment of an OLED display interface.

FIG. 15b shows a schematic diagram of an exemplary embodiment of an OLED board interface.

FIG. 16 shows a schematic diagram of an exemplary embodiment of an OLED 12V power supply for the wireless vital signs monitor.

FIG. 17 shows a schematic diagram of an exemplary embodiment of a membrane switch user interface showing simple intuitive control and GUI for the wireless vital signs monitor.

FIG. 18 shows a schematic diagram of an exemplary embodiment of a graphical user interface and membrane switch showing simplicity of functionality and color coded vital signs values based on user selectable alarm limits.

FIG. 19 shows a schematic diagram of an exemplary embodiment of a GUI for trend analysis of the wireless vital signs monitor transceiver unit showing scrolling window for care giver to search trends of individual vital signs.

FIG. 20 shows a schematic diagram of an exemplary embodiment of a GUI for transceiver unit showing individual wireless vital signs for the selected patient and interface switches for interfacing with casualty remotely.

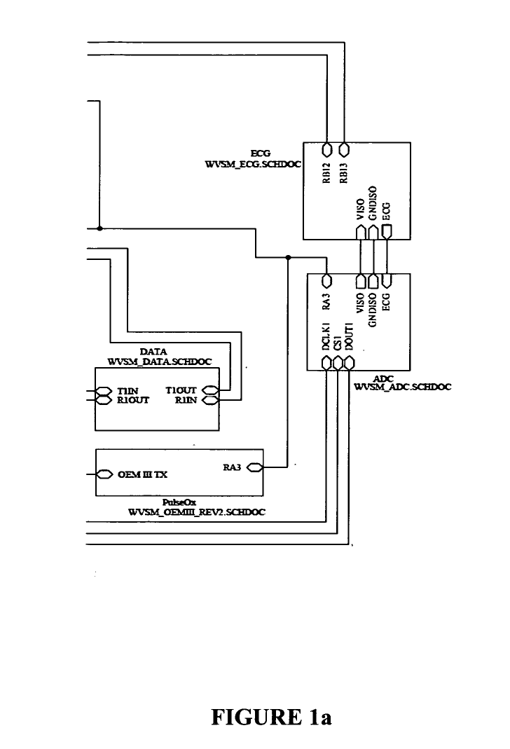

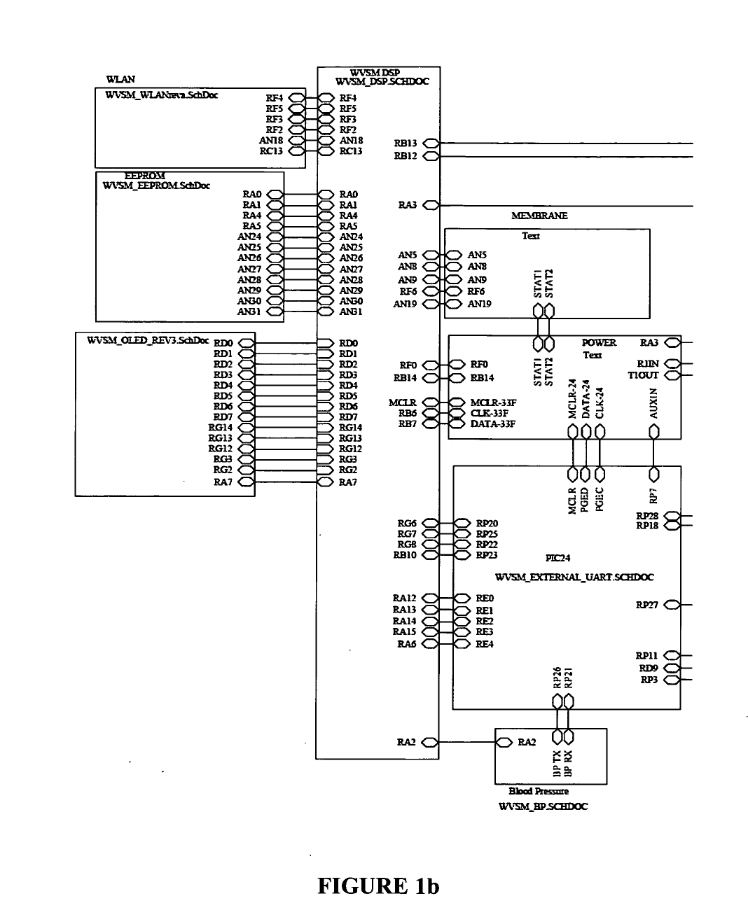

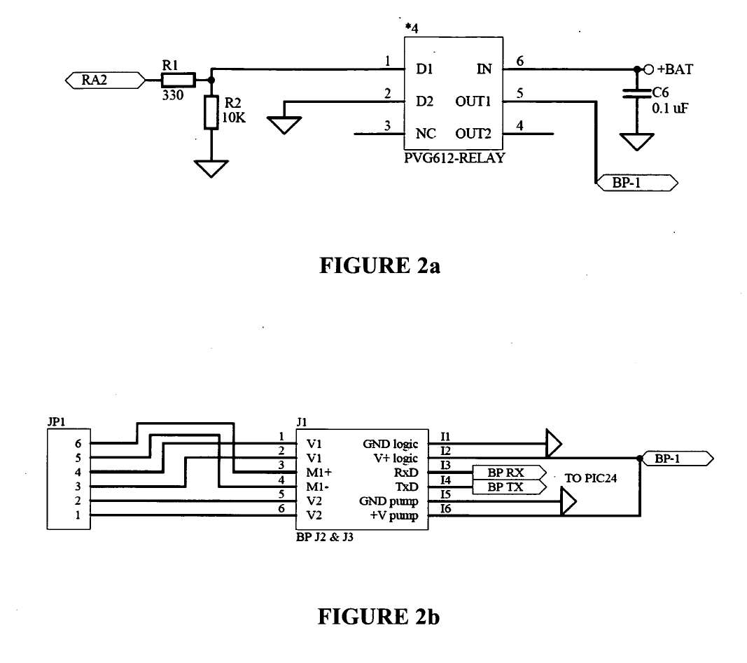

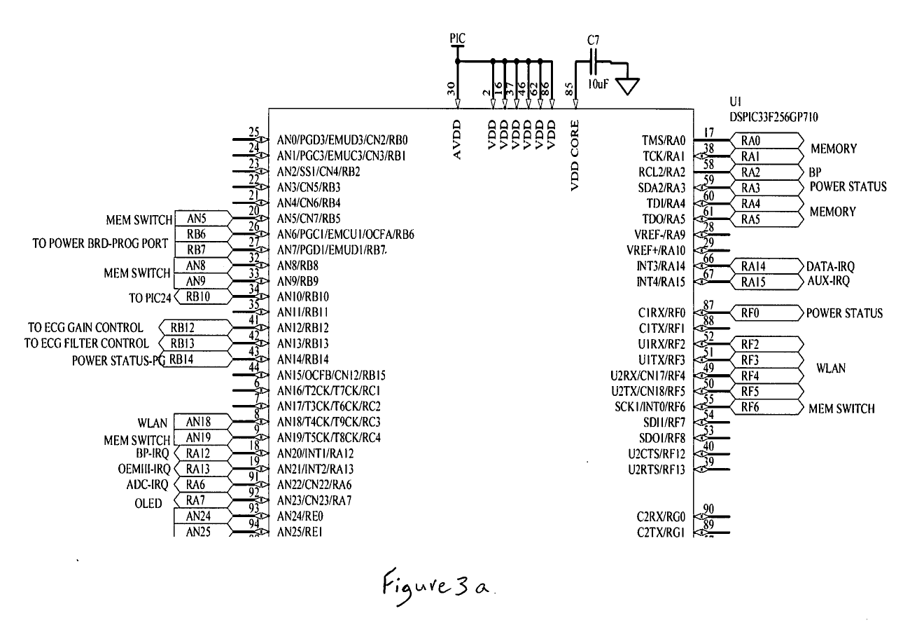

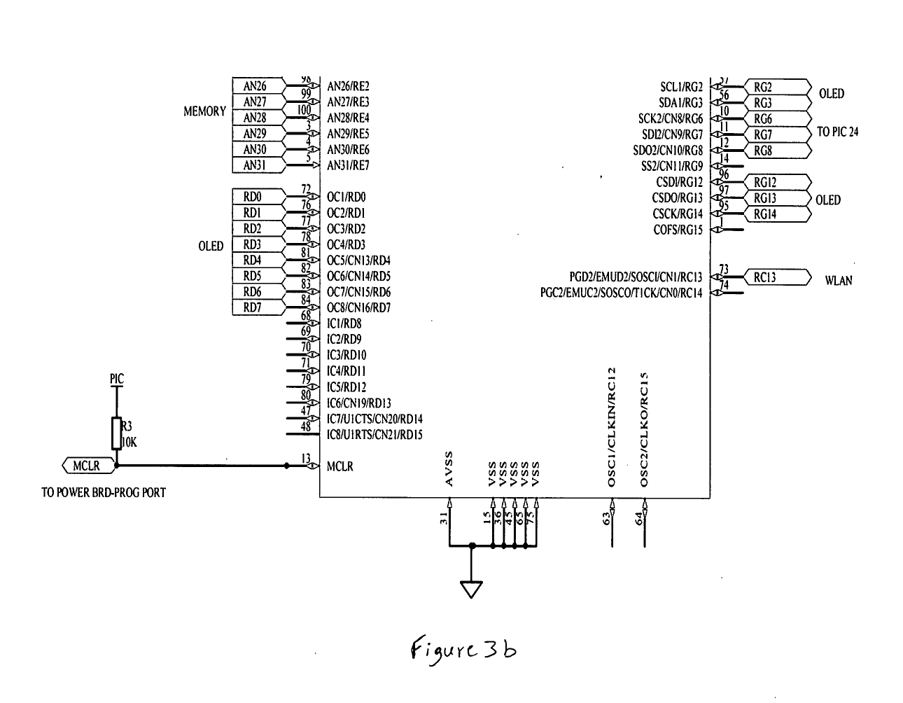

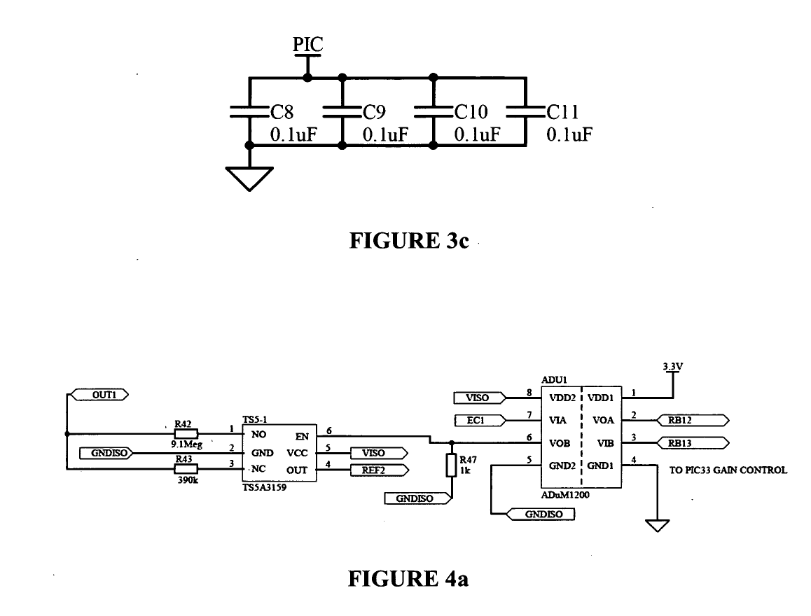

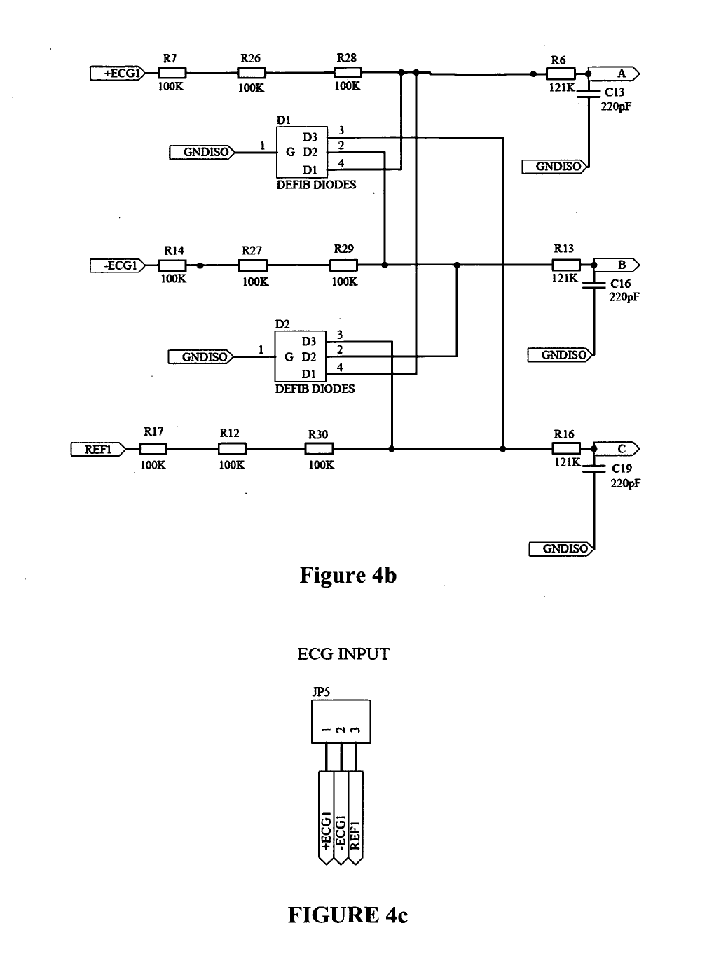

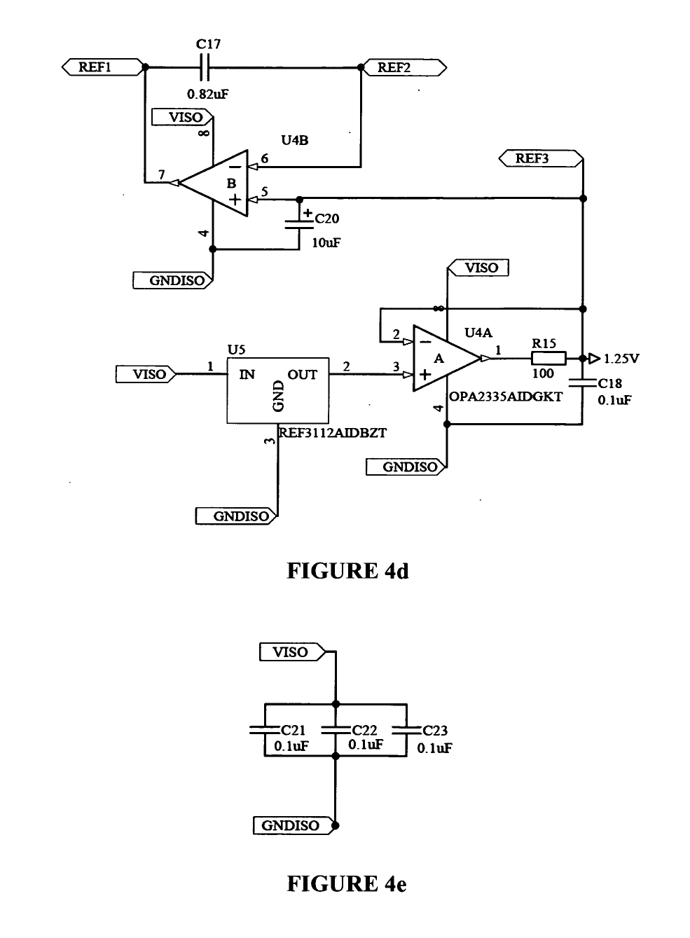

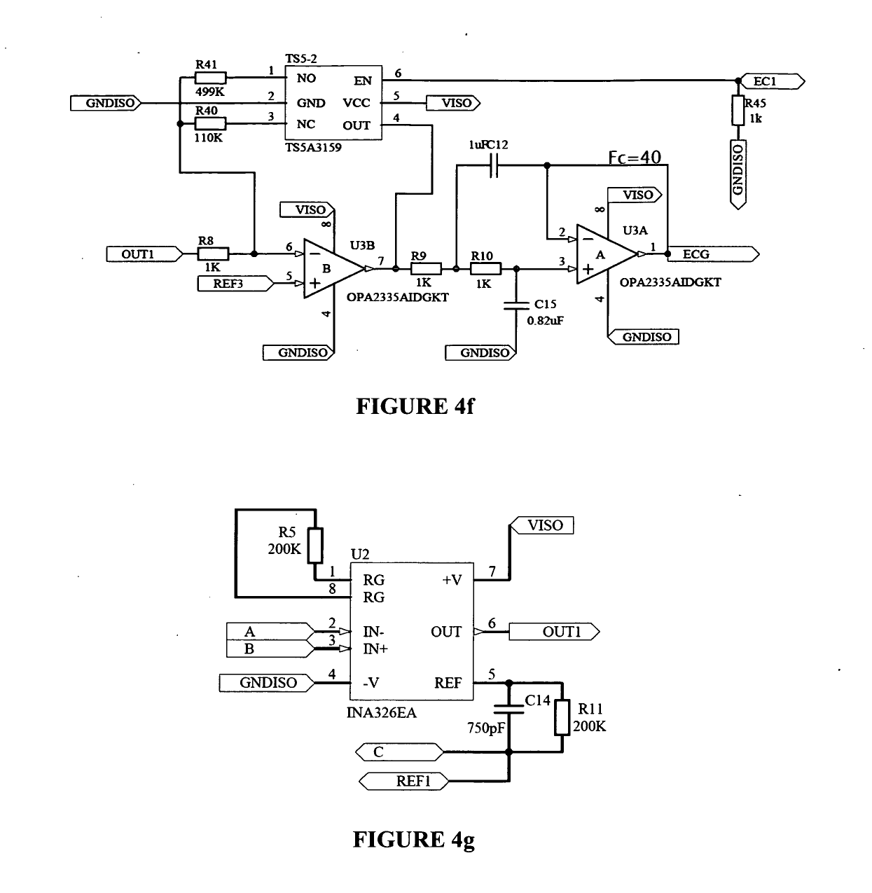

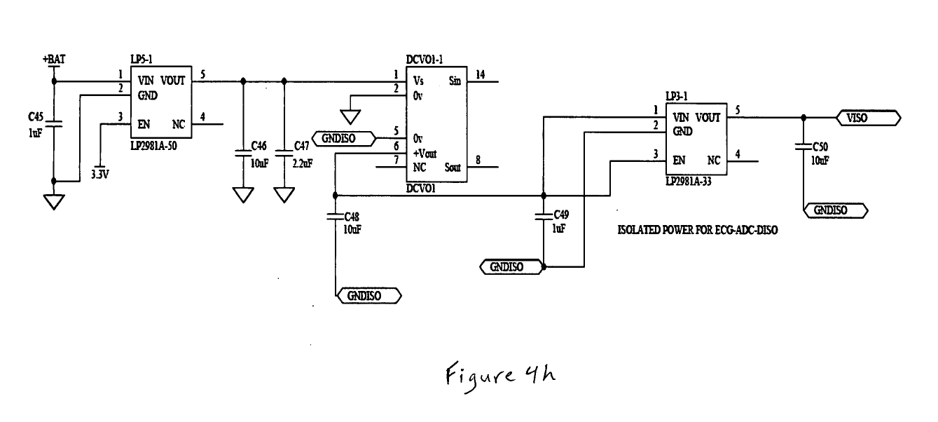

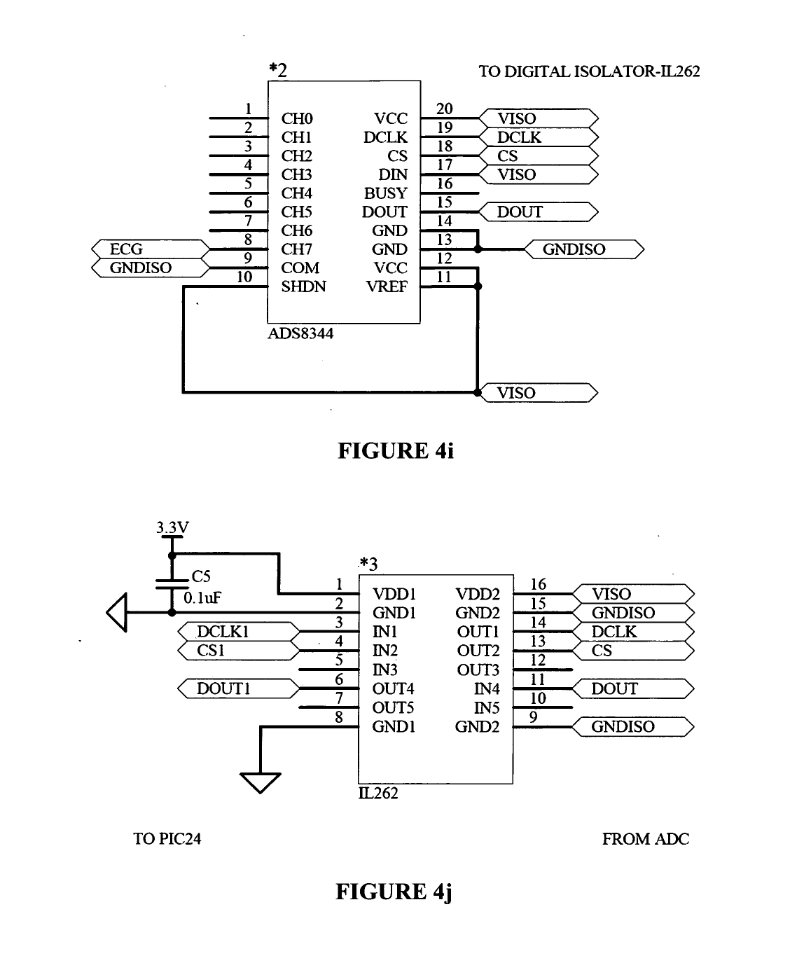

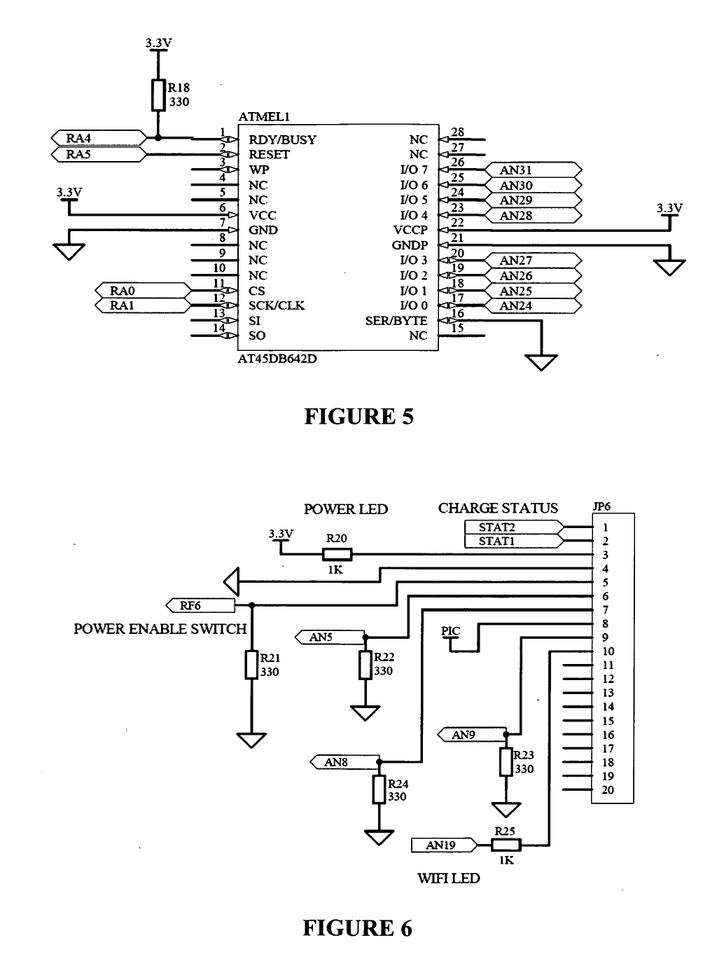

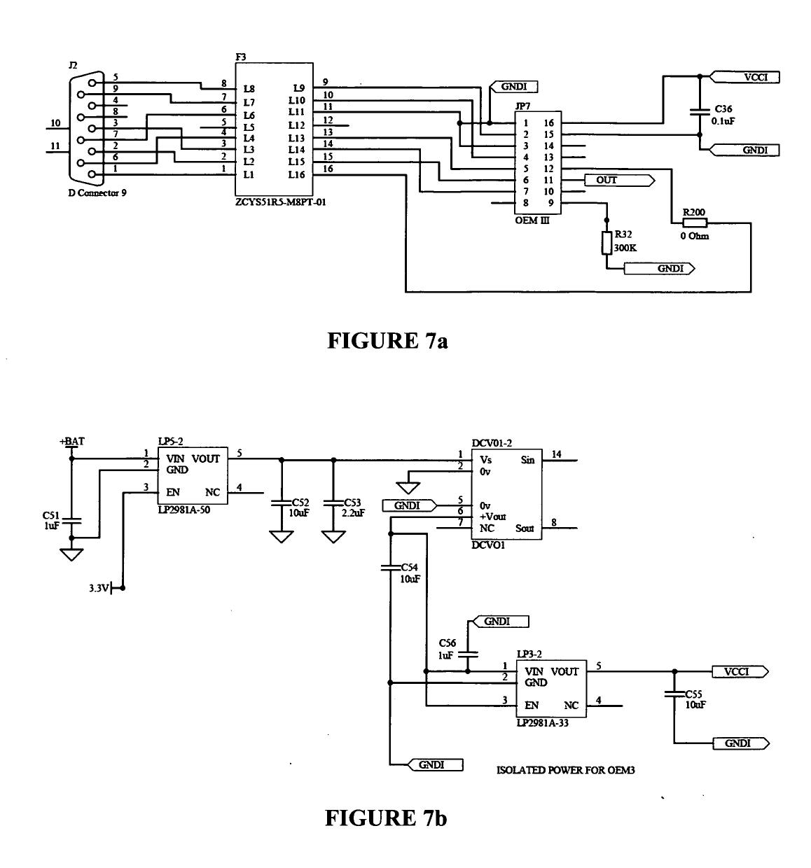

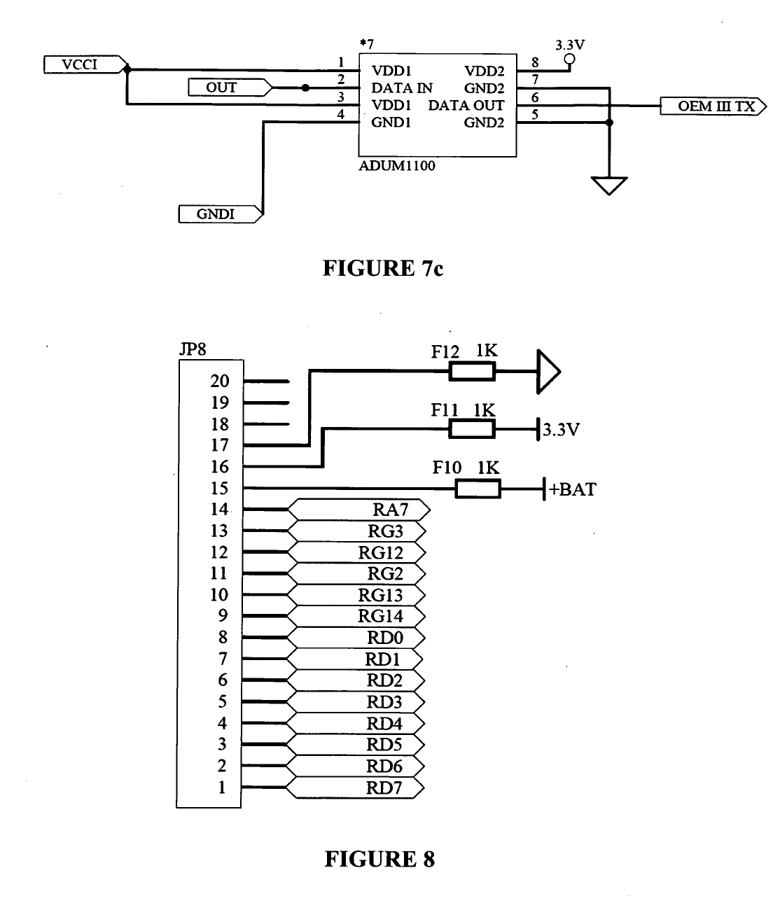

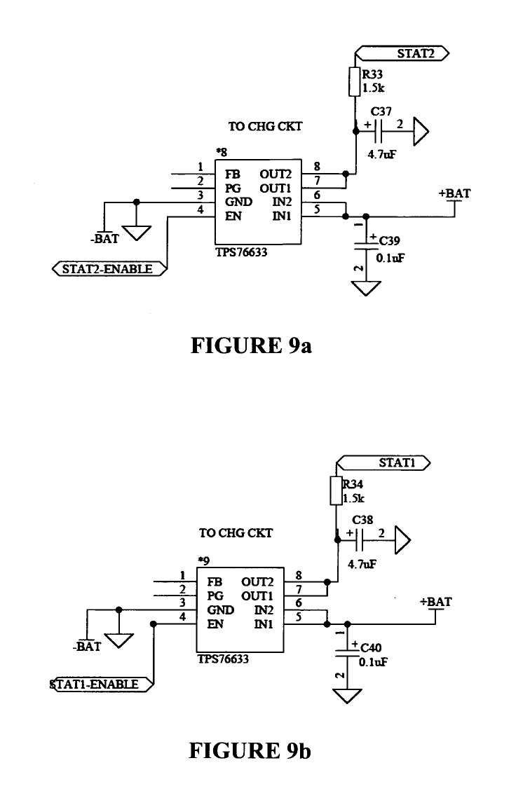

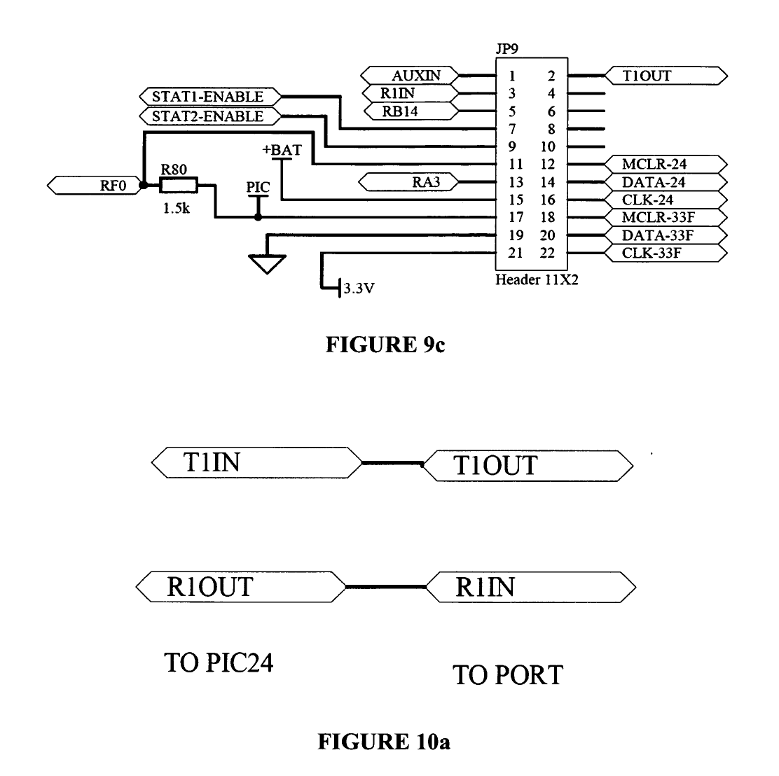

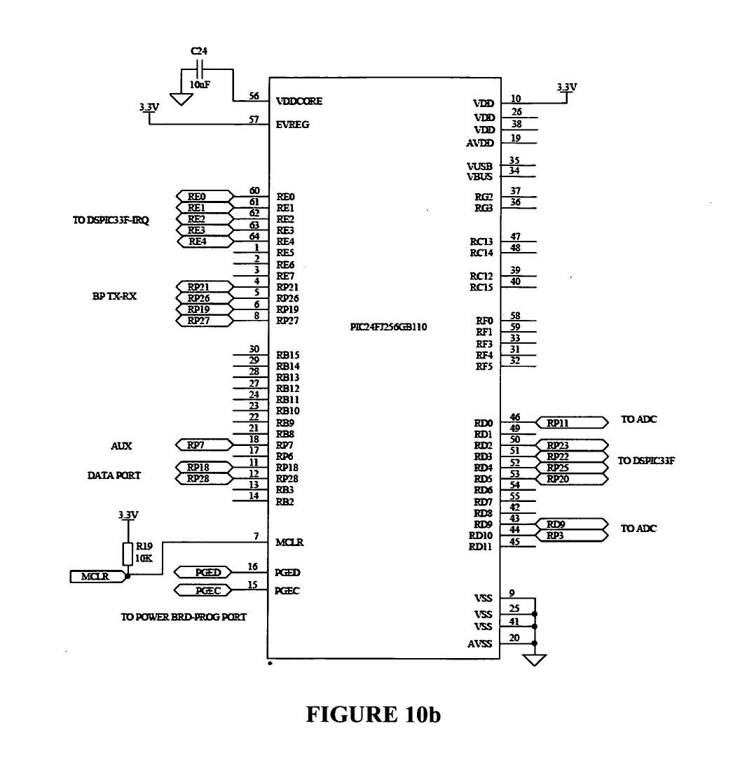

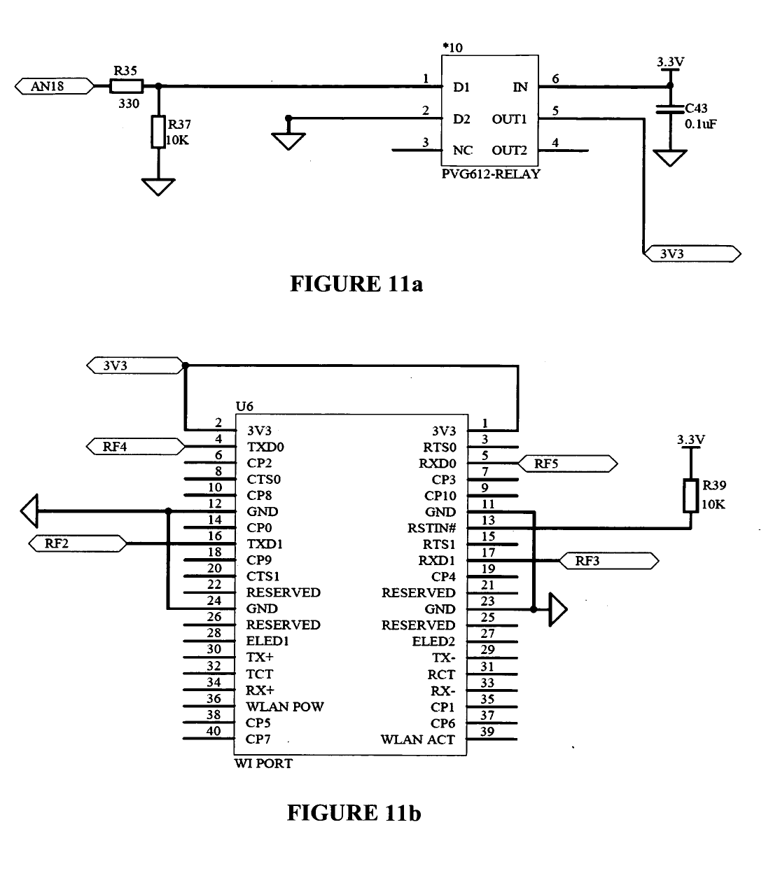

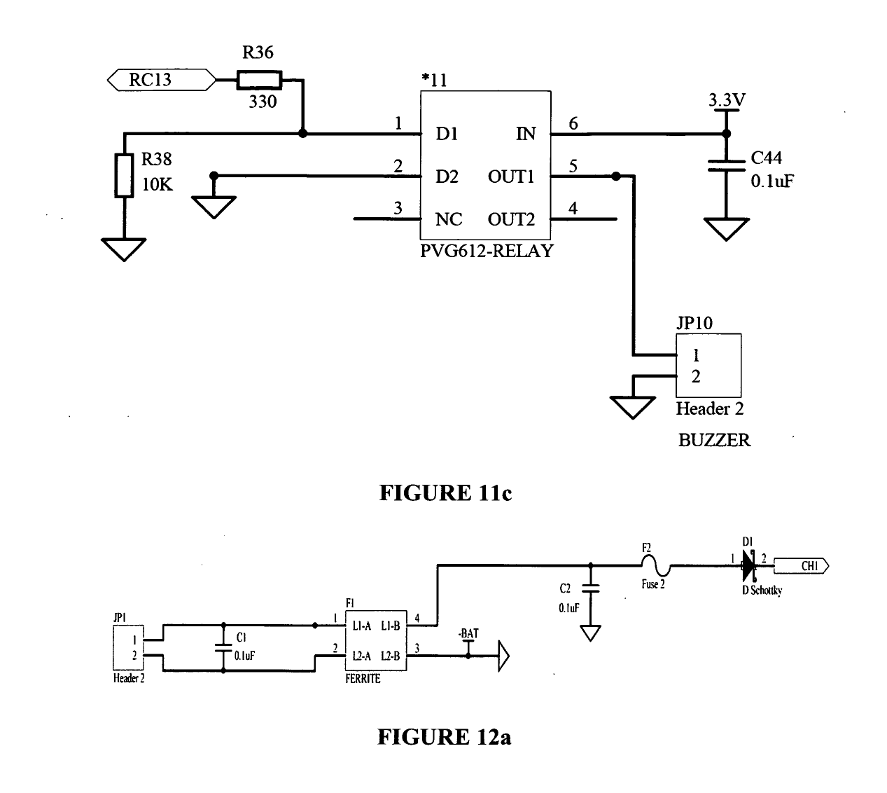

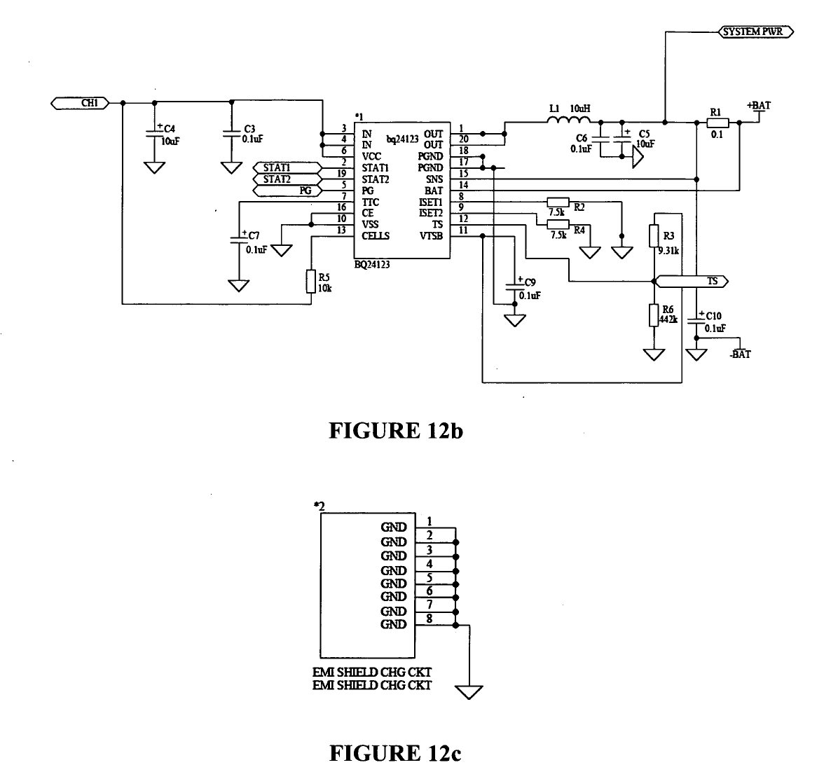

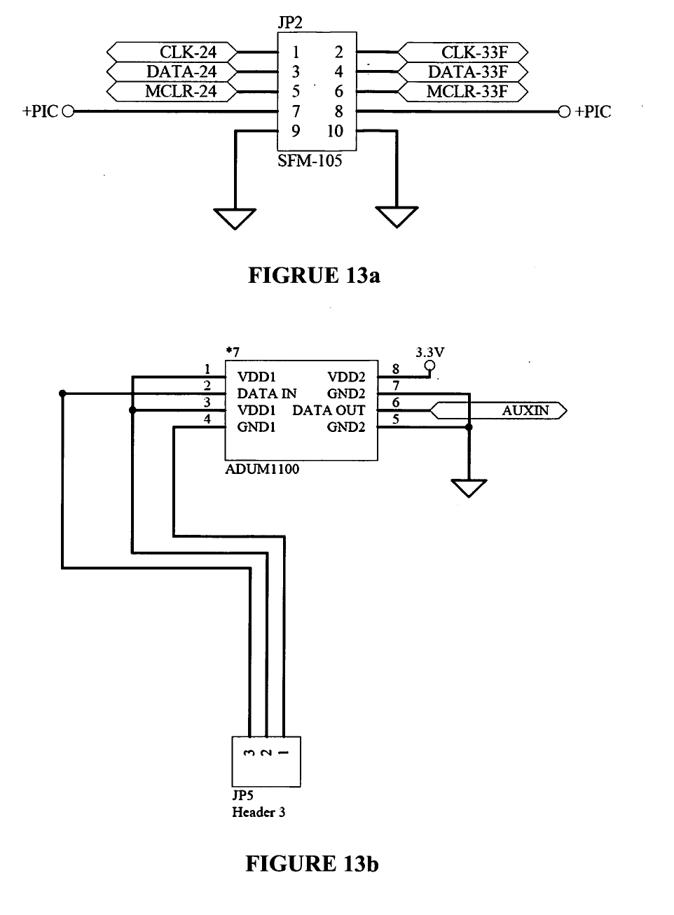

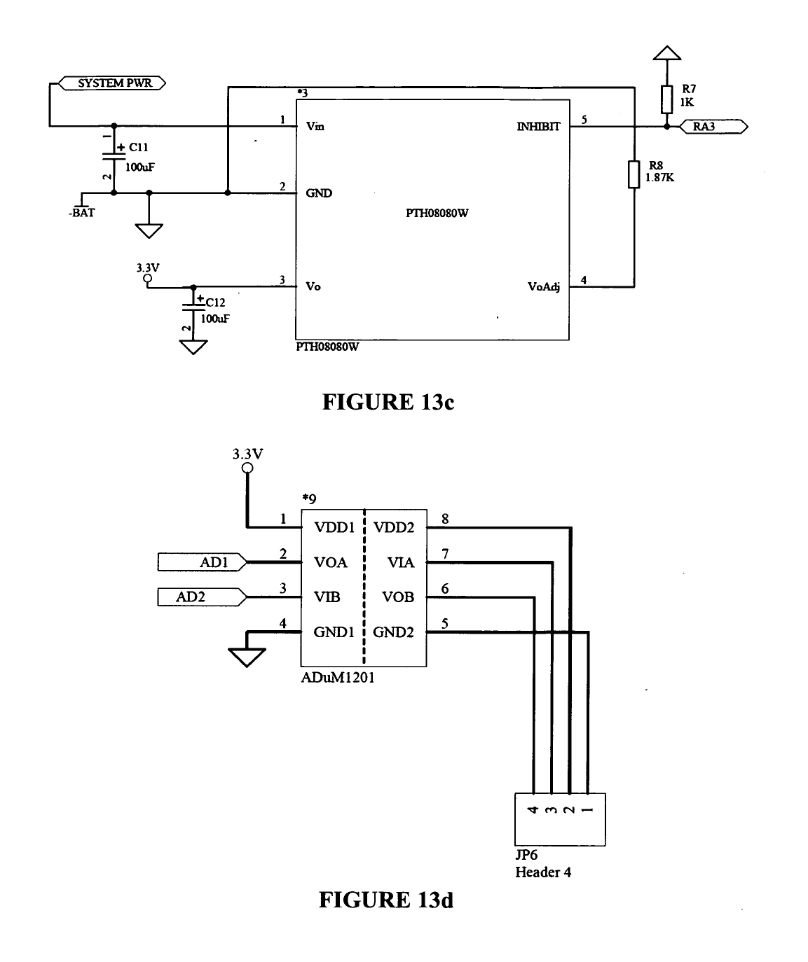

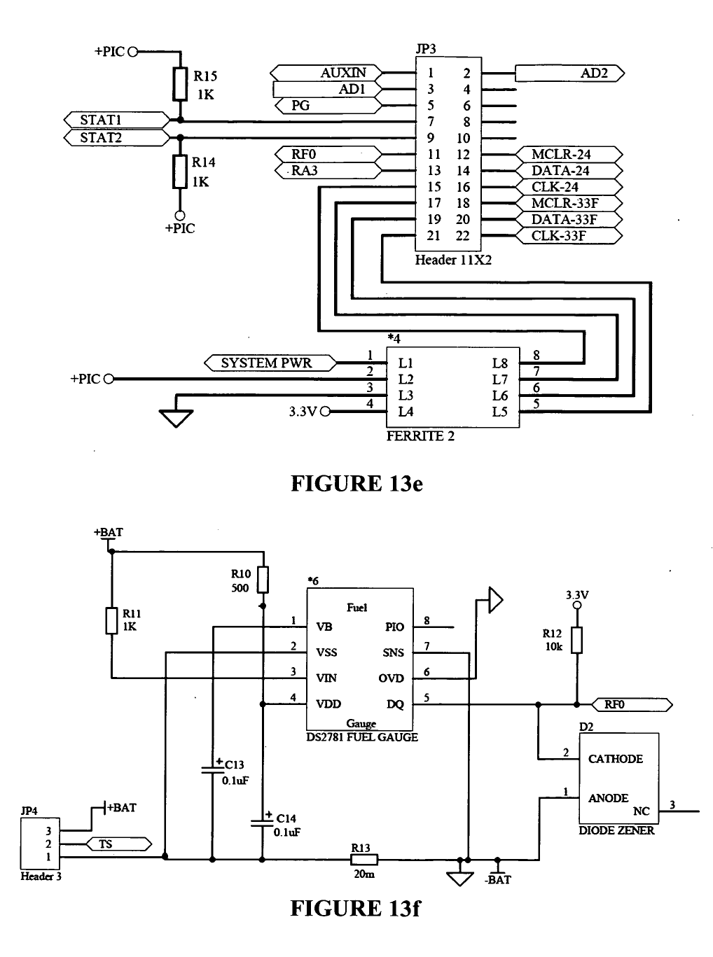

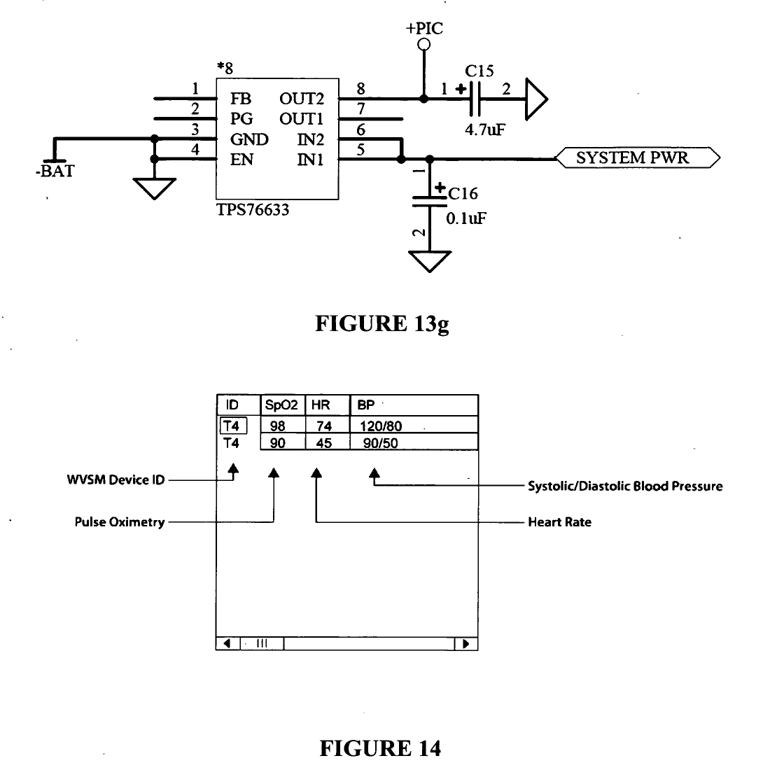

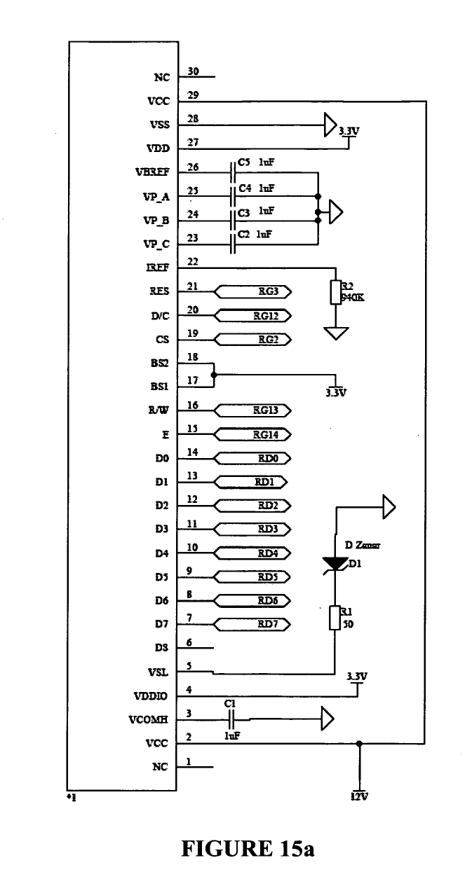

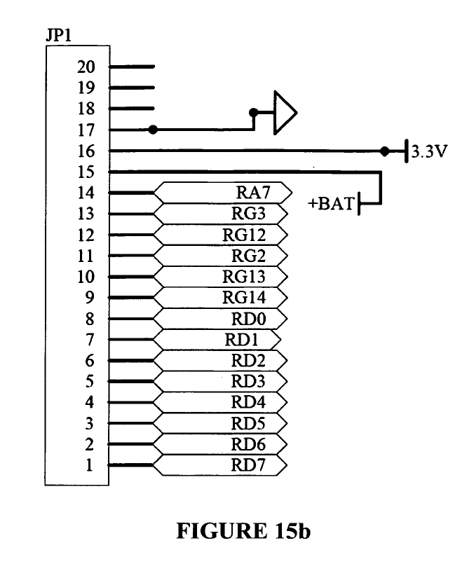

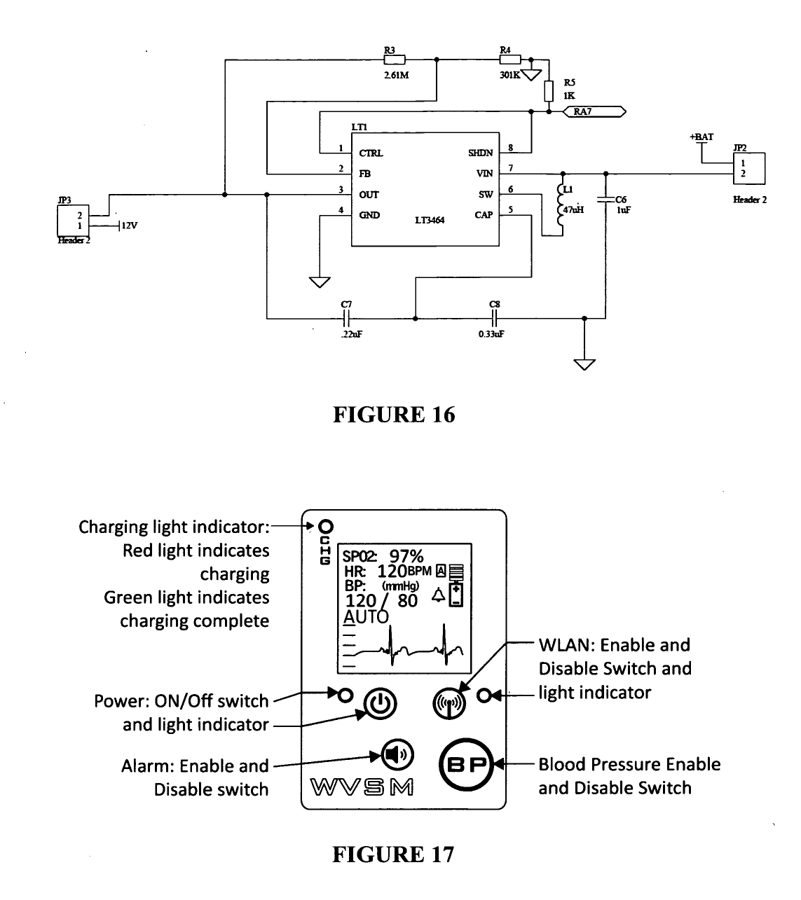

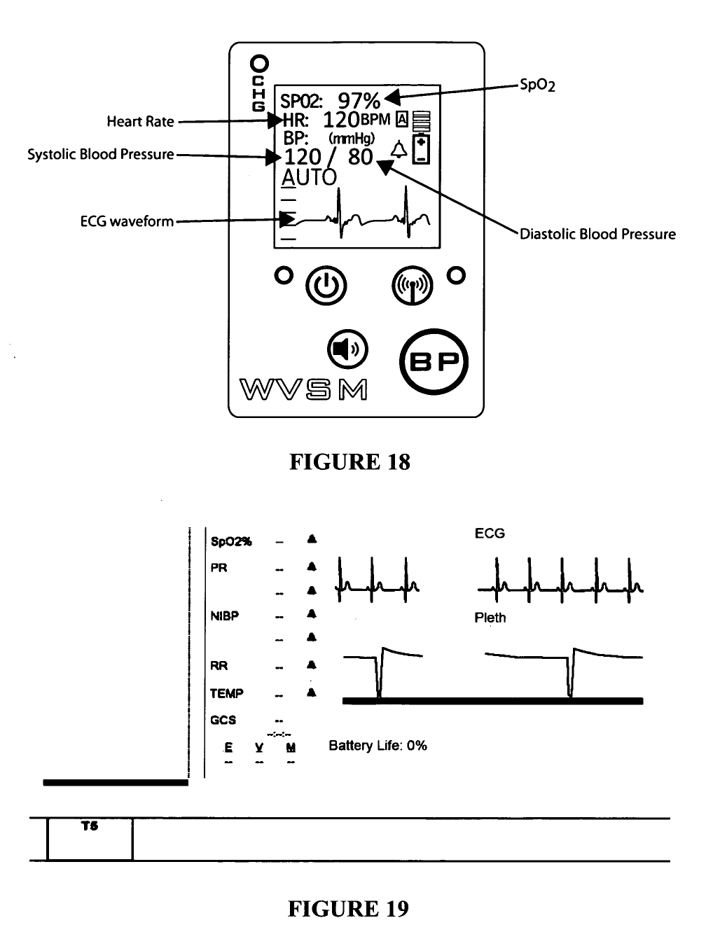

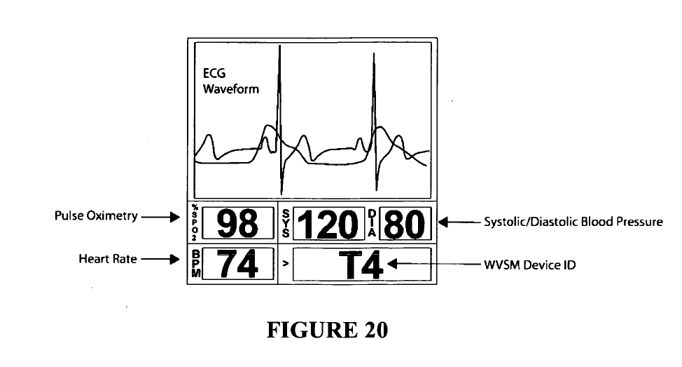

### DETAILED DESCRIPTION OF THE INVENTION

Further objectives and advantages of the present invention will become apparent as the description proceeds and when taken in conjunction with the accompanying drawings. To gain a full appreciation of the scope of the present invention, it will be further recognized that various aspects of the present invention can be combined to make desirable embodiments of the invention. Unless defined otherwise, all technical and scientific terms used herein have the same meaning as commonly understood by one of ordinary skill in the art to which this invention belongs. Where a term is provided in the singular, the inventor also contemplates the plural of that term. The nomenclature used herein and the procedures described below are those well known and commonly employed in the art.

### I. Device and System for Wireless Monitoring of the Vital Signs of Patients

One embodiment of the present invention includes an apparatus for wireless monitoring of the vital signs of a patient, including a plurality of patient mounted vital signs monitors, each having a plurality of sensors for detecting and measuring the vital signs of a patient. The present invention can provide a system whereby health care providers can maintain patient longitudinal situational awareness of data across the spectrum of health care providers. The sensors of the patient mounted vital signs monitors may include any desired and suitable sensor for the purpose the present invention and is contemplated for use. For example, the detecting and/or measuring sensors may include pulse waveform, skin temperature, skin humidity, multiple lead electrocardiograms, non-invasive blood pressure (NIBP), and saturation of blood oxygen. In addition the sensors may allow a specific and detailed calculation of multiple derived parameters through combinations and trends of current vital sign measures, for example, the plurality of sensor data may be processed to calculate additional vital signs data such as shock index, pulse wave delay as an indicator of changes in non-invasive blood pressure, heart rate complexity, and a summary alarm feature that indicates dynamically changing and overall vital signs state of the patient (called “Murphy Factor” or Life Saving Intervention (LSI) Probability).

The patient mounted vital signs monitor of the present invention also includes a first display for displaying the patient's vital signs, the display being operatively connected to the plurality of sensors, wherein the display can receive and display the patient vital signs. In such a configuration the patient mounted vital signs monitor may act as a stand alone display monitor for the vital signs of a patient, or not have any display and simply send the obtained data to the remote display to be observed, or both. The present invention can also transmit the patient's vital signs, for example by way of a transceiver, to remote locations, such as to emergency personnel that are on the scene tending to multiple patients, or to remote personnel able to connect into the wireless protocols available and linked to the system. As used herein, a “transceiver” is contemplated and may include a device that has both a transmitter and a receiver which are combined and share common circuitry or a single housing, and/or also may include a trans-receiver such as a device that no circuitry is common between transmit and receive functions. In such a configuration, the patient mounted vital signs monitor also includes a first transceiver operatively connected to the patient mounted vital signs monitor, and operable to transmit the patient's vital signs data to a remote transceiver by way of a first processing means for processing the detected or measured vital signs and for controlling the operation of at least the first display and the first transceiver automatically and autonomously. Such systems may include, for example, standard Wi-Fi® or Bluetooth® wireless communications hardware, and may use, for example, IEEE 802.11 or Bluetooth® chipsets. The transceivers of the present invention may include a combination transmitter/receiver in a single package, and may include full duplex capability which allows reception of signals during transmission periods.

In circumstances where the patient's vital signs are transmitted to a remote location or to emergency personnel on the scene, the present invention also includes a user mounted processing system and monitor platform having a second transceiver for wireless connection to the first transceivers of the plurality of the patient mounted vital signs monitors, and also a second display configured to separately display each of the patients' vital signs data received from the plurality of the patient mounted vital signs monitors by way of a second processing means for processing the received vital signs data from the plurality of the patient mounted vital signs monitors and for controlling the operation of at least the second display and the second transceiver. In such a configuration of the present invention, the user, which can be an emergency personnel, may remotely monitor the vital signs of multiple patients via the user mounted processing system and monitor, where as other emergency personnel without a user mounted processing system and monitor can monitor the patient's vital signs values, trend history, and status, for example, via a color coded (e.g., green-normal, yellow-caution, and red-serious) visual display by way of the patient mounted vital signs monitor. This configuration can be very efficient and effective in situations where a large number of injured patients are being treated by a small number of emergency personnel such as in the case of a disaster site or in a combat zone. Such an embodiment can allow for rapid triage based on the parameters shown using similar triage scoring of green, yellow, and red.

The present invention can be configured to send the vital signs of the patients (e.g., encrypted format) from the scene to other remote locations such as a nearby hospital or anywhere in the world providing that the receiving unit has, for example, internet connectivity and the system software. In such a configuration, the present invention may include a communications module that can be configured to be co-operable with at least one or more of the user mounted processing system and monitors, wherein the user mounted processing system and monitor can transmit the vital signs of the patients and the communication module can be configured to receive the vital signs data and be operable to transmit the vital signs data to a remote location anywhere in the world, for example using existing communications infrastructures automatically. For example, the user mounted processing system and monitor and/or the communication module may be provided with internet connectivity, and this connectivity can be used to transmit the data to any receiving station with internet connectivity via standard internet protocols.

In the present invention, the first display of the patient mounted vital signs monitor can be any suitable display for this purpose. In some embodiments of the present invention, it is preferable for the display of the patient mounted vital signs monitor to be comprised of a flexible display and/or modified sensor system that is configured to be placed on a patient and flex with the patient's body. In such configuration, depending on the contours of the patient's body or the location of the attachment of the monitor, the display can flex and bend accordingly.

The transceivers of the present invention can be any suitable transceivers that are suited for their desired purpose and the distances involved for transmitting and/or receiving the vital signs data and information. For example, the transceivers of the patient mounted vital signs monitor and the user mounted processing system and monitor can be configured to wirelessly communicate by way of IEEE 802.11b, 802.11g, Zigbee® 802.15.4, Bluetooth® 802.15.1, or standard off the shelf smart phones.

The monitors of the present invention may include other components and systems, for example, the display of the user mounted processing system and monitor may be configured to measure and display the range to the patient mounted vital signs monitors and producing one or more alarm when the patient mounted vital signs monitor is out of range of the user mounted second display, for example the alarms may be color coded to represent preset user alarm conditions and produce both visual and auditory alarms based on parameters, derived parameters, and trend-lines. An option to allow for vibration the hand held display units can also be embedded to alert the user to changes in patient status. Furthermore, in situations where a larger numbers of injured patients are being treated and monitored at the scene, the present invention can be configured to handle large numbers of patients, for example up to about 250 patient mounted vital signs monitors, but preferably between about 5 up to about 25 can be in wireless communication with the user mounted processing system and monitor. In addition, the user mounted processing system and monitor can further include a kill switch that can be configured to turn on and off the wireless communication to a particular patient mounted vital signs monitor. In some embodiments, the user mounted processing system and monitor can further include a patient locator which can be configured to transmit a pulsing display command to one particular or all patient mounted vital signs monitors that are in range. In other embodiments, all the patient data may be autonomously and wirelessly transferred from the patient mounted monitor to a base system, for example a hospital, once the patient is in range of the base system for storage and display of vital signs data. Furthermore, the hospital system can be configured to display all vital signs data and trending, and also further process vital signs data and calculate more complex derived vital signs, for example, a laptop or computer base system can generate the display of data and be configured to generate additional derived vital signs data such as predicting the need for a life saving intervention and heart rate variability, and/or display all of the vital signs data (sensor parameters and calculated vital signs) as well as data trends all on one display.

It is an important aspect of the present invention that, for example, all of the patient's data may be stored only on the patient mounted vital signs monitor and no long term data may be stored on the user mounted processing system and monitor. The data is stored on the patient mounted vital signs monitor, such as by utilizing non-volatile memory, and such data can be retrieved, for example, by using a wireless or wired serial connection to a PC with related software.

### EXAMPLES

### Example I

### Device and System for Wireless Monitoring of the Vital Signs of a Patient

One example of the wireless vital signs monitor (WVSM) is a highly mobile, patient-worn medical monitoring system using wireless Wi-Fi® 802.11b/g technology and standard off-the-shelf Windows® based software and hardware; it connects to multiple PCs, smart phones, and PDAs. WVSM allows medical personnel to be in touch with remote or on-site monitoring.

The WVSM is designed to attach directly onto the patient via standard blood pressure arm cuff for both sedentary and mobile patients. It integrates standard medical technologies such as the Nonin® SpO2 sensor, standard arm cuff NIBP, and multiple ECG configurations and connectors already on the market—thereby minimizing product acquisition and support costs. WVSM acquires motion tolerant electrocardiograms (at 230 Hz), SpO2, pulse rate and NIBP with selectable alarm limits and data rate capability. WVSM may be configured to process up to 254 simultaneous patients and can be embedded with algorithms to track real-time heart rate variability (rt-HRV), shock index, pulse wave delay (PWD), and pulse pressure in all patients.

All WVSMs use an on board OLED low power, full color, 128×128 resolution display showing patient HR, SpO2, and NIBP. User interface includes selectable press-and-release switches for starting/stopping NIBP, turning on and off the wife, and turning the unit on and off.

The WVSM can be configured to run wirelessly. PC and PDA enabling software is provided for full control of the WVSM within existing IT platforms, with no additional or hidden mandatory software purchases needed to implement. Additional vital signs can be manually entered such as the patients Glascoe Coma Score or GCS and patient respiration rate. Numerous WVSM-enabled patients can be linked together and displayed on common platforms. The WVSM is essentially a plug and play device requiring less than a minute to implement effectively in any environment at any time.

The WVSM basic monitoring system is comprised of the medical monitor WVSM unit, a non-invasive blood pressure cuff (NIBP) and lines, SpO2 sensor and connectors, and ECG leads and connectors. On the top of the WVSM are three standard plug-in leads that are color coded according to where on the body the lead sensors (stick-on) are placed. Red is left leg (LL), black is left arm (LA), and white is right arm (RA).

Manufacturer directions should be followed when placing the ECG leads on the body. The SpO2 connector (female) is on the front side of the WVSM. This connector is designed to accommodate the Nonin® SpO2 sensor and the attached finger clip as well as stick on sensors and reflectance forehead sensors. The NIBP connector is at the end of an approximate 6 inch pneumatic line from inside the WVSM device. The quick connector attaches firmly to a standard NIBP cuff with a quick insertion and snap turn.

The WVSM is a DC powered device and the Li-Polymer battery is internal to the device, and is accessible device only by disassembly of the device. The batteries do not need to be removed from the unit to be recharged.

The WVSM Unit can be operated by the following exemplary steps:

Step 1—Connect all applicable leads and sensors as needed on the unit and on the patient. Turn “ON” WVSM: The ON/OFF switch is located on the front face of the unit as it faces the user. Upon turning on the unit the green Power LED located on the front face will indicate that the unit is powered. The blue LED indicates WLAN activity and that the unit is automatically searching for a WLAN (802.11b/g) connection enabled with WVSM software. In addition, the display located in the center of the front surface of the WVSM unit indicates the vital signs SpO2, HR (heart rate), and BP (mm Hg) (Non-invasive Blood Pressure or NIBP in milliliters of mercury or mm Hg). The display will show dashed lines indicating no signal upon power up and then switch automatically to patient data as it begins to be obtained.

Step 2—The WVSM is set up to automatically take blood pressure every 15 minutes. To initiate an immediate blood pressure reading, simply press the BP button. To terminate a BP reading in progress, press the BP button.

The PDA can be operated by the following exemplary steps:

Step 1—Note: In order to communicate with the WVSM the PDA needs to be setup to operate on the same network. The units sent by the manufacturer are already configured properly. New systems should be set up by the user.

Step 2—Upon power up the screen on the PDA should present the display in standard MS Windows® format. It is now possible to engage WVSM transmittance into the PDA (or other device). Note: some PDAs and other mobile devices may vary in startup menus and icons.

Step 3—Touch Start on the Home Screen. The PDA will display typical menu icons. Find the File Explorer icon. Navigate to My Device\WVSM using the “Down” arrow in the upper left hand corner.

Step 4—Under My Device, Select “WVSM”.

Step 5—Select “WVSM.exe” program.

Step 6—The Main Screen appears. The software in the product allows registration of patients and selected functionality by attending medical personnel. Select “OK” to go directly to the monitoring screen.

Step 7—WVSM Patient List The WVSM software in the PDA automatically searches for and logs in the patient monitor when in range of the PDA. Typical indoor ranges are about 100 yards and 300 yards line of sight with substantial structure and reinforced concrete limiting wireless transmissions. All patients in range will produce patient icons displayed on the PDA. Verification is shown by the appearance of the patient ID (# of the WVSM unit, e.g., #W5) on the PDA patient list screen and vital signs for that patient. In this display all patients and data can be viewed simultaneously and reviewed. Users can access any of the registered patients by touching the patient number and data line to display that subjects' data in more detail and/or to affect alarm settings.

Each active WVSM monitor is displayed on the PDA screen in the order in which it was powered “ON” and subsequently connected, received, or “seen” wirelessly by the PDA. All patients connected to an active monitor will transmit patient data in color coded formats based on the vital signs alarm settings used (default values are pre-installed but can be adjusted in the alarm settings). Patient vital sign data within normal ranges, as set in the PDA, will display green highlighted data. When vital signs are either approaching or are out of line with alarm limits, the color changes from green to yellow to red highlights. In one application, if the unit is on, but no vital signs are connected to it, all data shows as Out of Track.

Step 8—WVSM Patient data-Main Patient Screen touching or tapping anywhere on the data line of a patient on the patient list screen will display specific vital signs for that patient as shown here. This same display reflects the previous step's “patient data” but adds full ECG waveform.

Each patient would display data here. The patient's registered unit is shown in the lower right corner, and last BP recorded (121/81), Heart Rate (76), SpO2 (96) and the lead 2 ECG are shown in real time based on sensor inputs.

Specifications for this WVSM system include:

### Microprocessor

High performance 40 MIPS DSP and MCU core

128K program memory, 16 KB RAM, and 64 KB Flash

16-bit ADC producing up to 1 KHz sampling rates

### Analog Features

Patient and system electrical isolation

High performance biomedical instrumentation amplifier

### On Board Display

Low power, passive matrix OLED

128×128 resolution with 16 bit color

25×25 mm viewing area

### Power Management

Standard 7 hours battery life

Auto-sleep for power conservation

### Wireless

Network Interface Wireless 802.11b/g; 328 foot range indoors, 900 ft+range LOS

Multiple protocols supported in 802.11b/g (ARP, UDP, TCP, Telnet, ICMP, SNMP, DHCP, BOOTTP, Auto IP)

Media Access Control CSMA/CA with ACK

Security Password protection with locking features, 64/128 bit WEP, TKIP

### Sensors

ECG at 230 Hz, Channel-user selectable time window with embedded software digital filtering and high GAIN

SpO2/HR via Nonin® OEM III module

Oxygen Saturation Range 0 to 100%

Pulse Rate Range 18 to 300 pulses per minute

Rate Accuracy 3%±1

Blood pressure, NIBP; Common Cuff, Range: Systolic: 40 mmHg to 260 mmHg, Diastolic: 20 mmHg to 200 mmHg, heart rate range: 40 BPM to 200 BPM and Serial Output

### Graphical User Interface/Software Compatibility

All connectivity software included

User selectable and auto peer-to-peer or multiple access point connectivity

Compatible with Windows® XP

Wi-Fi® enabled (fixed and mobile devices)

User ID, patient ID utilizing selectable patient alarms, data storage with post processing pulse pressure, heart rate variability (from R wave), and shock index.

Across entire patient database from remote positions. Software installed for remote consult data capture on patient records.

Architecture allows interfacing to peripheral devices in both I/O configurations via serial interface or other wireless protocols.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims. Although the invention has been described and illustrated with respect to exemplary embodiments thereof, it should be understood by those skilled in the art that the foregoing and various other changes, omissions, and additions may be made therein and thereto, without parting from the spirit and scope of the present invention.

What is claimed is:

1. An apparatus for wireless monitoring of the vital signs of one or more patients simultaneously, comprising:
a plurality of patient mounted vital signs monitors, each comprising:
a plurality of sensors for detecting and measuring the vital signs of a patient;
a first display for displaying the patient's vital signs, the display being operatively connected to the plurality of sensors, wherein the display can receive and display the patient vital signs;
a first transceiver operatively connected to the patient mounted vital signs monitor, and operable to transmit the patient's vital signs data to a remote transceiver; and
a first processing means for processing the detected or measured vital signs and for controlling the operation of at least the first display and the first transceiver;
a user mounted or carried processing system and monitor comprising:
a second transceiver for wireless connection to the first transceivers of the plurality of the patient mounted vital signs monitors;
a second display configured to separately display each of the patients' vital signs data received from the plurality of the patient mounted vital signs monitors; and
a second processing means for processing the received vital signs data from the plurality of the patient mounted vital signs monitors and for controlling the operation of at least the second display and the second transceiver;
whereby, users can monitor and record current and history of the vital signs of a plurality of patients simultaneously by way of the patient mounted vital signs monitor or by way of the user mounted display.

2. The apparatus of claim 1, further comprising a communications module co-operable with at least the user mounted processing system and monitor to receive the vital signs data and configured to transmit the vital signs data to a remote location.

3. The apparatus of claim 1, wherein the plurality of sensors comprises sensors for electrocardiogram, non-invasive blood pressure, heart rate, pulse waveform, respiration rate, and saturation of blood oxygen.

4. The apparatus of claim 1, wherein the plurality of sensor data are processed to calculate additional vital signs data such as shock index, pulse integrity, pulse wave delay, cardiac rhythm complexity, and heart rate variability as an indicator of changes in non-invasive blood pressure, autonomic nervous system integrity, and a summary alarm feature that indicates overall vital signs state of the patient.

5. The apparatus of claim 1, wherein the first display of the patient mounted vital signs monitor comprises a flexible or fixed display disposed to be placed or stuck on a patient and flex with the patient's body.

6. The apparatus of claim 1, wherein the first and the second transceivers wirelessly communicate by way of IEEE 802.11 b or 802.11 g, or 802.15.4 Zigbee, Bluetooth 802.15.1, or a smart phone.

7. The apparatus of claim 1, wherein the second display of the user mounted processing system and monitor is configured to measure and display the range to the patient mounted vital signs monitors and produce one or more alarms when the patient mounted vital signs monitor is out of range of the user mounted second display.

8. The apparatus of claim 7, wherein the one or more alarms are color coded to represent preset alarm conditions and produce both visual and auditory alarms based on parameters, derived parameters, patient history, and trendlines.

9. The apparatus of claim 1, wherein between about 1 up to about 250 patient mounted vital signs monitors are in wireless communication with the user mounted processing system and monitor.

10. The apparatus of claim 1, wherein the user mounted processing system and monitor further comprises a kill switch that is configured to turn on and off the wireless communication to a particular patient mounted vital signs monitor.

11. The apparatus of claim 1, wherein connectivity can be switched from wireless to wired.

12. The apparatus of claim 1, wherein the user mounted processing system and monitor further comprises a patient locator that is configured to transmit a pulsing display command to one particular or all patient mounted vital signs monitors that are in range.

13. The apparatus of claim 1, wherein all of the patient's data is stored on the patient mounted vital signs monitor and no long term data is stored on the user mounted processing system and monitor.

14. The apparatus of claim 1, wherein all of the patient data is autonomously and wirelessly transferred from the patient mounted monitor to a base system once the patient is in range of the base system for storage and display of vital signs data.

15. The apparatus of claim 14, wherein the user can input directly additional information, patient vital signs, and indices which are then stored for further and subsequent display.

16. The apparatus of claim 14, wherein the base system comprises a laptop or computer that is configured to generate the display of data and is configured to generate additional derived vital signs data such as predicting the need for a life saving intervention and heart rate variability.

17. The apparatus of claim 16, wherein the laptop or computer is configured to display all the vital signs data from sensor parameters calculated vital signs as well as data trends all on one display.
